(19) United States
(12) Patent Application Publication
(10) Pub. No.: US 2014/0147814 A1
(43) Pub. Date: May 29, 2014

(54) BONE GRAFT CONTAINMENT DEVICES

(71) Applicant: Zimmer, Inc., Warsaw, IN (US)

(72) Inventors: Michael Scott Collins, San Marcos, CA (US); Sarah Zimmerman, Columbia City, IN (US); Donald L. Yakimicki, Warsaw, IN (US); Daniel P. Murphy, Claypool, IN (US); Dean M. Acker, Napels, FL (US)

(73) Assignee: Zimmer, Inc., Warsaw, IN (US)

(21) Appl. No.: 14/091,650

(22) Filed: Nov. 27, 2013

Related U.S. Application Data
(60) Provisional application No. 61/731,199, filed on Nov. 29, 2012.
(60) Provisional application No. 61/846,327, filed on Jul. 15, 2013.

(51) Int. Cl. A61F 2/28

(52) U.S. Cl. CPC A61F 2/2803
USPC 433/215

(57) ABSTRACT

A flexible containment device can comprise a textile material including at least one of a woven material, a braided material, a knit material, a felt material, and an electrospun material, wherein the textile material includes a plurality of biocompatible strengthening fibers configured to engage a bone graft material and configured to remain in a patient.

### CLAIM OF PRIORITY

This application claims the benefit of U.S. Provisional Patent Application Ser. No. 61/731,199, filed on Nov. 29, 2012, and also claims the benefit of U.S. Provisional Patent Application Ser. No. 61/846,327, filed on Jul. 15, 2013, the benefit of priority of each of which is claimed hereby, and each of which are incorporated by reference herein in its entirety.

### TECHNICAL FIELD

The present disclosure relates to bone grafts and, more specifically, to bone grafts in the oral, maxillofacial region.

### BACKGROUND

In dental applications, it can be important to improve or create bone for implantation of dental devices such as dental implants. Tooth loss or decay can cause bone loss or decay and in many instances, bone areas must be improved before any prosthetic devices can be implanted to take the place of lost or damaged teeth. Current dental grafting options work reasonably well in creating horizontal bone (bone perpendicular to the axis of a tooth). However, vertical bone growth (that is, parallel to the axis of the tooth) remains challenging. A major reason for this difficulty is that soft tissue can fill in the area where bone growth is desired. Once soft tissue has filled this space, bone will not grow into the desired area.

An area of the body typically needing bone grafts prior to implant placement is the crestal ridge or alveolar ridge on the mandible and maxilla. Existing methods of improving bone in this area include using a bone graft material such as allografts, autografts and synthetic grafts, and containing the bone graft material with a metal mesh containment device or a membrane containment device. The metal mesh may require removal after a certain period of time and in many instances the removal can disturb or destroy any new bone growth. Removal of a mesh device can also cause soft tissue trauma. A containment device with a membrane material such as collagen may not be strong enough to resist crushing forces in the mouth area while the bone graft heals to normal bone strength. Further, metal mesh devices are not easily cut or shaped and a dentist or surgeon may be required to keep a large inventory of shapes and sizes on hand to effectively treat patients.

Another problem encountered in this field concerns a rate of graft turnover. Graft turnover is the replacement of bone graft material with living cells from the patient. In a high turnover rate, the graft material is resorbed too quickly and soft tissue can invade the graft area. Bone growth will not occur where the soft tissue has developed. If the graft material has a turnover rate that is too low, osseointegration issues may develop and the healing time between placing the bone graft and installing a dental implant becomes too long or delayed. Dentists and oral surgeons completing oral bone grafts need a material that is strong enough to resist crushing forces, yet flexible, formable, and shapeable enough so that they can quickly produce a graft containment device tailored to a particular patient and location.

### OVERVIEW

There exists a need for a flexible bone graft containment device that has sufficient strength to maintain its shape and resist crushing forces while a bone graft is healing, and yet have enough flexibility to make insertion easier and allow the device to be cut to size during surgery. Textiles can offer the ability to significantly customize the properties of a graft containment device and provide sufficient flexibility while maintaining sufficient strength for a bone graft containment device. These properties can be customized to optimize soft tissue and bone tissue growth in the desired implantation areas. Although the flexible containment device is described herein for placement in a mouth of a patient, other designs of the flexible containment device can be used in other parts of the body. Given the composition of the device, it can remain in the body and does not have to be removed.

Containment devices having textile portions can include woven, braided, or knitted materials. Containment devices having textile portions can also include non-woven materials such as electrospun material or felted material. Further, containment devices having combinations of the foregoing textile portions are also possible.

The fibers of the textile portion can be single filament or multi-strand yarn formed from various materials including but not limited to metals such as stainless steels, titanium, titanium alloys, and nitinol; polymers such as polyether ether ketone (PEEK), polyethelene, poly(methyl methacrylate) (PMMA), polyester, polytetrafluoroethylene (PTFE); and resorbable materials such as poly-L-lactide (PLLA), polyglycolic acid (PGA), and hydrogels.

A flexible containment device can include a combination of strengthening materials and resorbable materials. The strengthening materials can include metals or polymers and can provide strength or stiffness to resist deformation forces. Resorbable materials can be selected from a wide range of materials such as such poly-L-lactide (PLLA), polyglycolic acid (PGA), or hydrogels. A flexible textile bone graft containment device can be configured such that it does not need to be removed after placement in a patient.

To better illustrate the flexible containment device and methods disclosed herein, a non-limiting list of examples is provided here:

In Example 1, a flexible containment device can comprise a textile material including at least one of a woven material, a braided material, a knit material, a felt material, and an electrospun material, wherein the textile material includes a plurality of biocompatible strengthening fibers configured to engage a bone graft material and configured to remain in a patient.

In Example 2, the flexible containment device of Example 1 can optionally be configured such that the textile material includes a woven material.

In Example 3, the flexible containment device of any one or any combination of Examples 1-2 can optionally be configured such that the textile material includes a braided material.

In Example 4, the flexible containment device of any one or any combination of Examples 1-3 can optionally be configured such that textile material includes a knitted material.

In Example 5, the flexible containment device of any one or any combination of Examples 1-4 can optionally be configured such that the strengthening fibers are metal fibers and wherein the textile material includes the metal fibers in a range of about 20% to about 60% by weight.

In Example 6, the flexible containment device of Example 5 can optionally be configured such that the metal fibers comprise at least one of a stainless steel, titanium, titanium alloy, and nitinol.

In Example 7, the flexible containment device of any one or any combination of Examples 1-4 can optionally be configured such that the strengthening fibers are polymer fibers and wherein the textile material includes the polymer fibers in the range of about 20% to about 60% by weight.

In Example 8, the flexible containment device of Example 7 can optionally be configured such that the polymer fiber is comprised of at least one of polyether ether ketone (PEEK), polyethelene, poly(methyl methacrylate) (PMMA), polyester, and polytetrafluoroethylene (PTFE).

In Example 9, the flexible containment device of any one or any combination of Examples 1-4 can optionally be configured such that the strengthening fibers includes a metal fibers in the range of about 10% to about 40% by weight and polymer fibers in a range of about 10% to about 40% by weight.

In Example 10, the flexible containment device of any one or any combination of Examples 1-4 can optionally be configured such that the strengthening fibers include metal fibers in a range of about 20% to about 60% by weight and polymer fibers in a range of about 20% to about 60% by weight.

In Example 11, the flexible containment device of any one or any combination of Examples 1-10 can optionally be configured such that the fibers of the textile material comprise single strand fibers.

In Example 12, the flexible containment device of any one or any combination of Examples 1-11 can optionally be configured such that the fibers of the textile material comprise multi-strand fibers.

In Example 13, the flexible containment device of any one or any combination of Examples 1-12 can optionally be configured such that the textile material is configured to be placed in and remain in a mouth of the patient.

In Example 14, a flexible containment device can comprise a textile material including at least one of a woven material, a braided material, a knit material, a felt material, and an electrospun material, wherein the textile material includes a plurality of strengthening fibers, a plurality of resorbable fibers and a plurality of biocompatible fibers configured to engage a bone graft material and configured to remain in a patient.

In Example 15, the flexible containment device of Example 14 can optionally be configured such that the textile material is configured to be placed in and remain in a mouth of the patient.

In Example 16, the flexible containment device of any one or any combination of Examples 14-15 can optionally be configured such that the plurality of strengthening fibers include at least one of stainless steel, titanium, titanium alloys, nitinol, polyether ether ketone (PEEK), polyethelene, poly(methyl methacrylate) (PMMA), polyester, and polytetrafluoroethylene (PTFE).

In Example 17, the flexible containment device of any one or any combination of Examples 14-16 can optionally be configured such that the plurality of resorbable fibers are comprised of at least one of as poly-L-lactide (PLLA), polyglycolic acid (PGA), and hydrogels.

In Example 18, a method of bone grafting can comprise the steps of: cutting a gingival layer; exposing a bone surface; decorticating the bone surface; packing bone graft material into the bone surface; covering the bone graft material with a flexible containment device, the flexible containment device formed by at least one of braiding, weaving, knitting, felting and electrospinning, the flexible containment device further including a bone graft facing surface coated with a bone growth inducing substance; and suturing the cut gingival layer.

In Example 19, the method of Example 18 can optionally be configured such that the flexible containment device is comprised of a plurality of metal fibers.

In Example 20, the method any one or any combination of Examples 18-19 can optionally be configured such that the flexible containment device is comprised of a plurality of metal fibers and a plurality of polymer fibers.

In Example 21 the flexible containment device and the bone method of any one or any combination of Examples 1-20 can optionally be configured such that all elements, operations, or other options recited are available to use or select from.

### BRIEF DESCRIPTION OF THE DRAWINGS

Corresponding reference characters indicate corresponding parts throughout the several views. The exemplifications set out herein illustrate exemplary embodiments of the invention, and such exemplifications are not to be construed as limiting the scope of the invention in any manner.

FIG. 1 illustrates an oral site with a bone loss area.

FIG. 2 illustrates decortication of a bone loss area.

FIG. 3 illustrates application of a bone graft material to a bone loss area.

FIG. 4 illustrates covering a bone graft with a containment device.

FIG. 5 illustrates a plain weave textile containment material as constructed in accordance with at least one example.

FIG. 6 illustrates a satin weave textile containment material as constructed in accordance with at least one example.

FIG. 7 illustrates a twill weave textile containment material as constructed in accordance with at least one example.

FIG. 8A illustrates braided textile containment material as constructed in accordance with at least one example.

FIG. 8B illustrates braided textile containment material as constructed in accordance with at least one example.

FIG. 8C illustrates braided textile containment material as constructed in accordance with at least one example.

FIG. 9A illustrates knitted textile containment material as constructed in accordance with at least one example.

FIG. 9B illustrates knitted textile containment material as constructed in accordance with at least one example.

FIG. 10 illustrates a felt textile containment material as constructed in accordance with at least one example.

FIG. 11 illustrates an electrospun textile containment material as constructed in accordance with at least one example.

FIG. 12 illustrates a ridge splitting procedure in accordance with at least one example.

FIG. 13 illustrates a braided flexible containment device as constructed in accordance with at least one example.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

Disclosed herein is a flexible bone graft containment device and method. As outlined in the OVERVIEW section and described in further detail below, the flexible containment device can include numerous configurations. These configurations are exemplary in nature and are not intended to limit the spirit and scope of the present disclosure. Thus, numerous other configurations are also contemplated. A flexible containment device can include portions that include textiles. Textiles can take many forms such as woven, braided, knitted, and non-woven and can be comprised of multiple fibers of single or multiple strands.

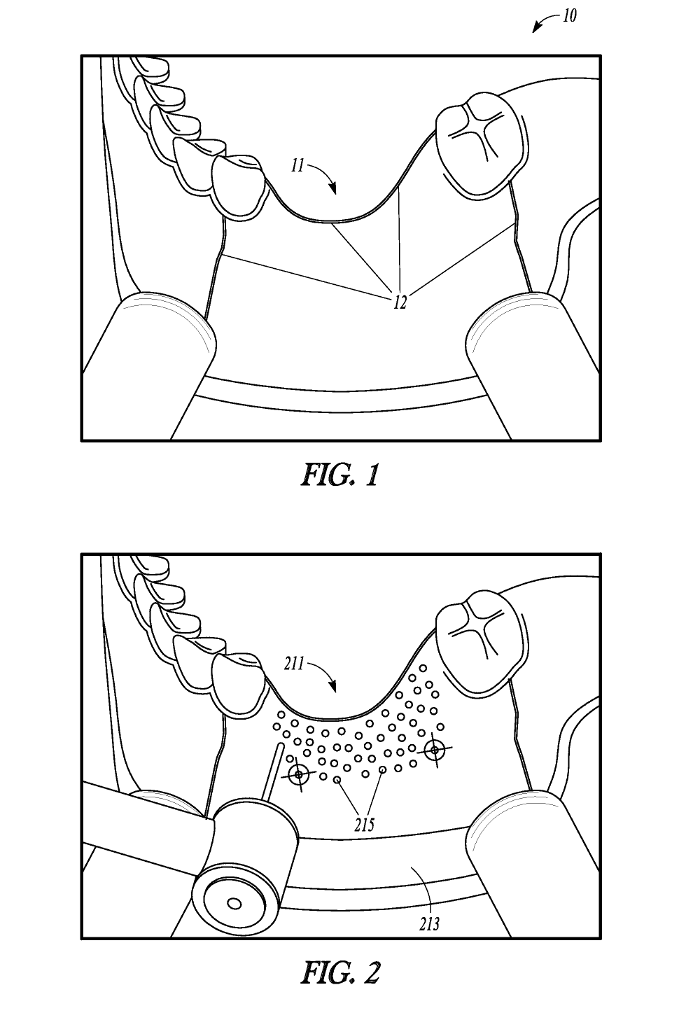

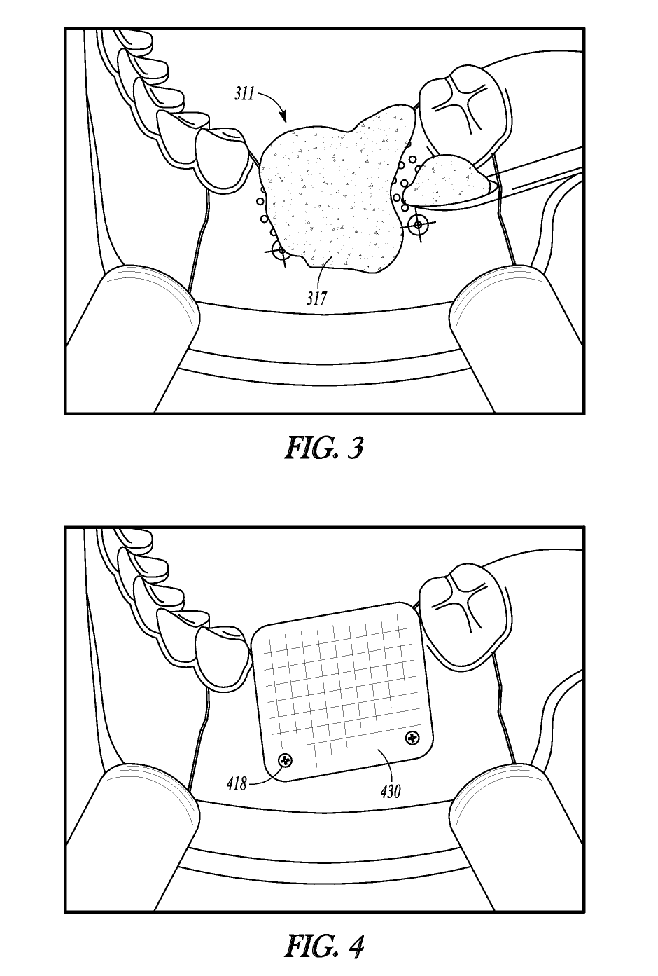

FIG. 1 illustrates an oral site 10 with a bone loss area 11. One or more gingival incisions 12 can be made to reveal the underlying bone structure. FIG. 2 illustrates the manner in which gingiva 213 can be peeled back out of the way and the bone loss area 211 can be decorticated with a series of drilled holes 215. The decortication can lessen healing time and improve bone graft strength. FIG. 3 illustrates an exemplary bone graft material 317 being packed into the bone loss area 311. FIG. 4 illustrates an exemplary flexible containment device 430 being applied so as to at least partially cover the bone graft material 317 (see FIG. 3). The flexible containment device 430 can be secured with one or more biocompatible fasteners 418. Because every patient can have a different oral geometry, and every bone graft procedure can require different containment needs, such as the size and shape of the containment area, flexible containment devices can be cut to a particular size and shape. Textiles can make up portions of a flexible containment device. Textile forms and materials can be pre-formed into a specific shape, such as a shape that can conform to the bone loss area 311 (see FIG. 3). Textile forms can be shaped by a surgeon at the surgical site and can have shape setting functions built in to the material used for the fibers, such as a shape setting polymer. A shape setting function can be built in to the textile form, such as a braided or woven textile that can be formed to a desired shape and can retain the new shape. Shape setting can be controlled by other parameters such as temperature. In an example, a polymer fiber can be used in the textile that will become more rigid when heated to a temperature in the range of body temperature.

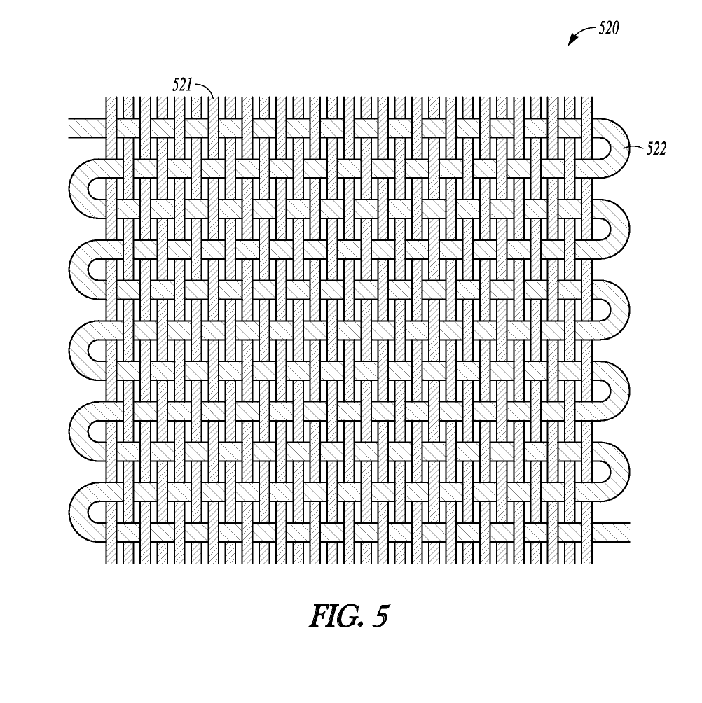

A flexible containment device can be manufactured using any textile form and combinations of textile forms. Weaving is a method of textile production in which two distinct sets of yarn or thread are interlaced at right angles to form a fabric or cloth textile. FIG. 5 illustrates an example of a plain weave 520 in accordance with at least one example of the present disclosure. The plain weave 520 can include longitudinal threads called warp fibers 521 and lateral threads called weft fibers 522. In a plain weave 520 each weft fiber 522 crosses the warp fiber 521 in an alternating “over-under” fashion.

Woven patterns can be varied in many ways. The density (number of fibers per unit of measurement) of either the warp fiber 521 or weft fibers 522, or both sets of fibers, can be altered. In FIG. 5 the weft fiber 522 is shown as continuous, but in another example, the weft fiber 522 can be made up of a plurality of fibers of varied material, type, or size. The warp fiber 521 can also be made up of a plurality of fibers of varied material, type, or size. Materials making up the fibers of the flexible containment device can be a single strand filament or multi-strand fiber including but not limited to metals, such as stainless steels, titanium, titanium alloys, and nitinol; polymers such as polyether ether ketone (PEEK), polyethelene, poly(methyl methacrylate) (PMMA), polyester, polytetrafluoroethylene (PTFE); and resorbable materials such as poly-L-lactide (PLLA), polyglycolic acid (PGA), and hydrogels. These materials can be combined in any manner to create a textile of the desired properties.

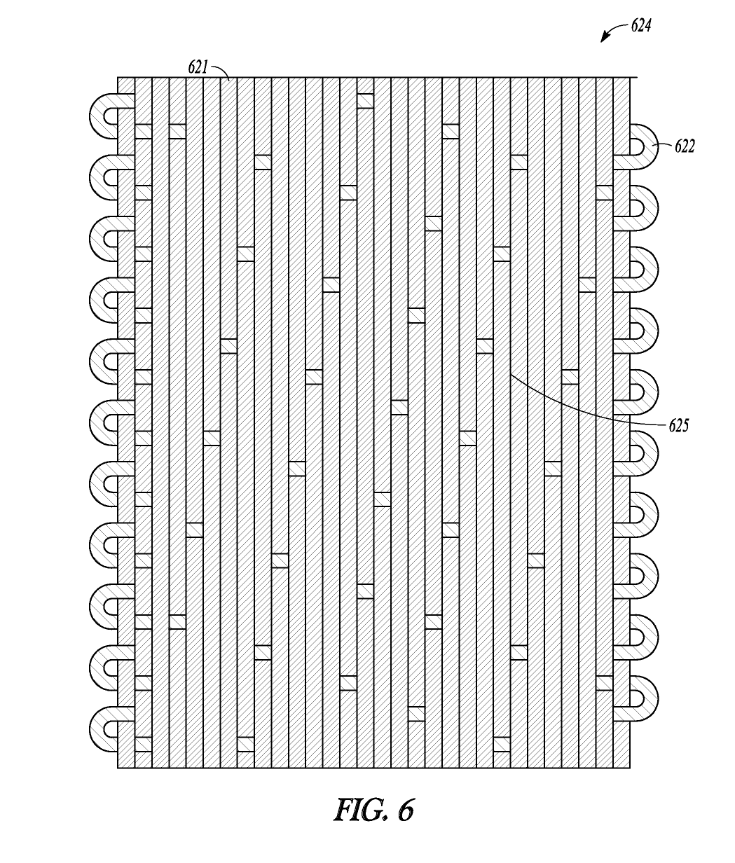

FIG. 6 illustrates an example of a satin weave 624 in accordance with at least one example of the present disclosure. The satin weave 624 is characterized by four or more warp fibers 621 floating over a weft fiber 622 or vice versa. In the illustrated example, the float area 625 of the warp fiber 621 floats over 16 weft fibers 622. The number of fibers that are floated over can vary and the weaving pattern does not need to be uniform or repeating. In an example, each warp fiber 621 can have a different float value than an adjacent fiber. In an example, the weft fibers 622 can all be metal fibers and the warp fibers 621 can all be made of a polymer. In an example, every other warp fiber 621 can be a metal fiber and the remaining warp fibers 621 can be made of a polymer. In various examples, all or portions of the woven fibers can be metal fibers, resorbable fibers, polymer fibers, or combinations thereof.

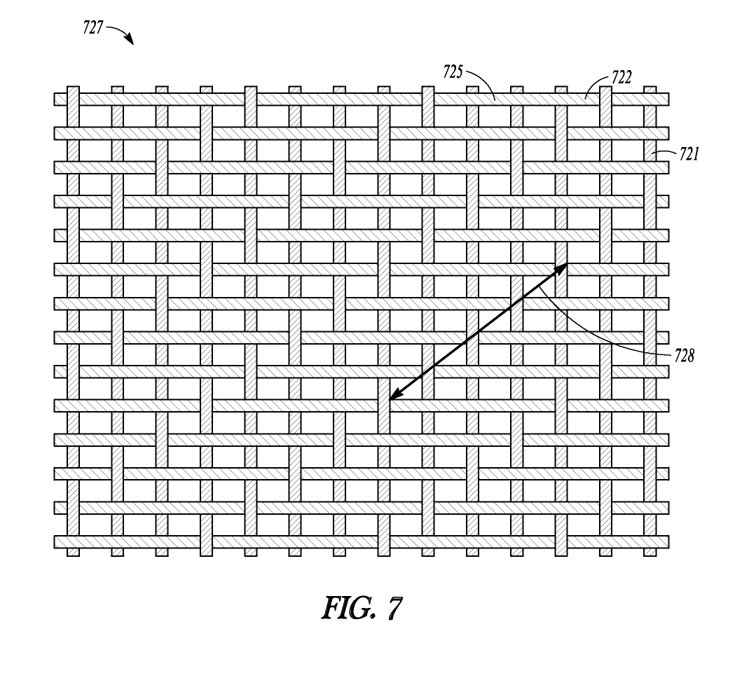

FIG. 7 illustrates an example of a twill weave 727 in accordance with at least one example of the present disclosure. A twill weave 727 is a type of textile weave with a pattern of diagonal parallel ribs (in contrast with a satin weave and a plain weave). The diagonal pattern is accomplished by passing the weft fiber 722 over one or more warp fibers 721 and then under two or more warp fibers 721 and so on, with a “step” or offset between rows to create a characteristic diagonal pattern. The diagonal line formed in this type of pattern is also known as a wale 728. The number of fibers that are floated at 725 in a twill weave 727 can vary.

As will be appreciated by those of ordinary skill in the art, FIGS. 5-7 illustrate basic weave types, and the weaving can be infinitely varied. Thus, the foregoing illustrations are provided merely for purposes of example and not limitation, and they are not intended to limit the scope and breadth of the flexible containment devices described herein.

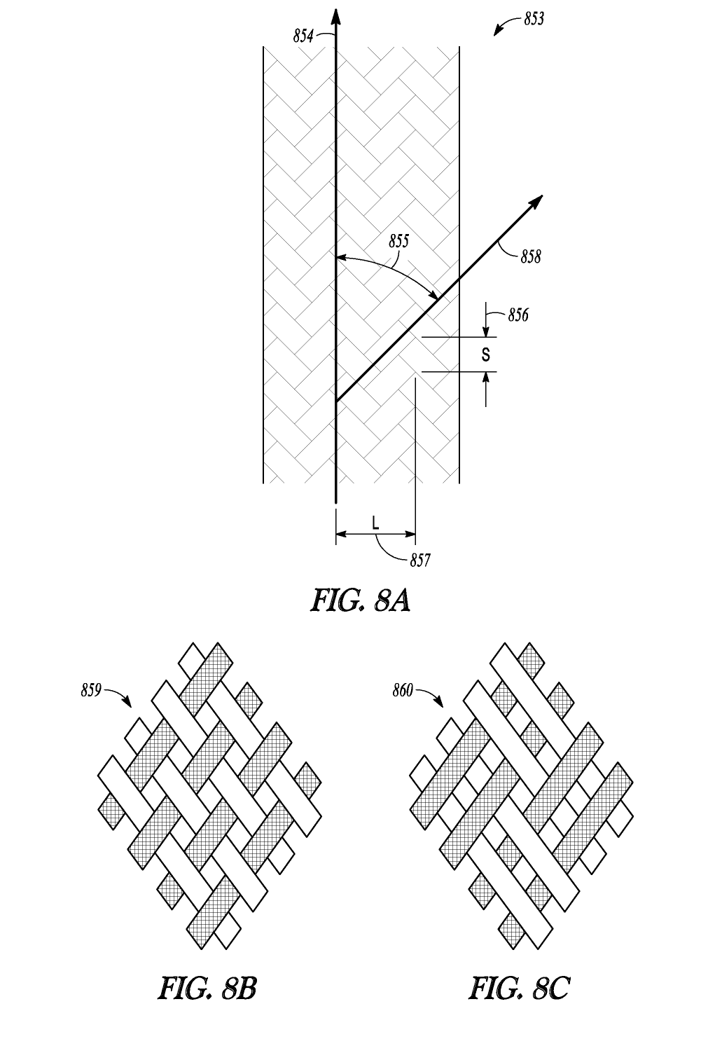

A textile device can alternatively or additionally include braiding 853, as illustrated in FIG. 8A. Braiding is a complex structure or pattern formed by intertwining three or more strands of fibers. Compared to the process of weaving (see FIGS. 5-7) which can be a wide sheet of textile from two separate, perpendicular groups of fibers (warp and weft), a braid can be long and narrow, with each component fiber “zigzagging” forward through the overlapping mass of the other fibers. More complex braids can be constructed from an arbitrary number of fibers to create a wider range of structures such as ribbon-like bands, hollow or solid cylindrical cords, or broad mats which resemble a rudimentary perpendicular weave. Braiding can create a textile product that involves the interlacement of fibers in a diagonal formation or bias 858, as illustrated in FIG. 8A. A braid axis 854 can be formed in the longitudinal direction defined by the mass of fibers. An angle formed between the braid axis 854 and the bias 858 is called a braid angle 855. The braid angle 855 can be varied to change the characteristics of the textile. The braid angle 855 can be varied from, for example about 10 degrees to about 85 degrees. Generally, a higher braid angle provides more longitudinal stiffness to the textile. Braiding 853 can be differentiated by the number of lateral repeating units 856 per unit measurement (called “picks” at “S”) and/or the number of repeating longitudinal units 857 (called “lines” at “L”) per unit measurement. Braiding 853 can be varied in many ways, for example in FIG. 8B illustrates a 1/1 pattern 859 where a fiber extending in a first direction crosses over and under fibers extending in a second direction in an alternating manner. FIG. 8C illustrates another example, a 2/2 pattern 860 can be formed by having a group of two fibers extending in a first direction cross over and under two fibers extending in a second direction in an alternating manner. These patterns can be uniform and repeating or variable. A textile can be braided in three dimensions. As in the woven textile materials, a braided flexible containment device can include various sizes and types of fibers and can include mixtures of sizes and types of fibers.

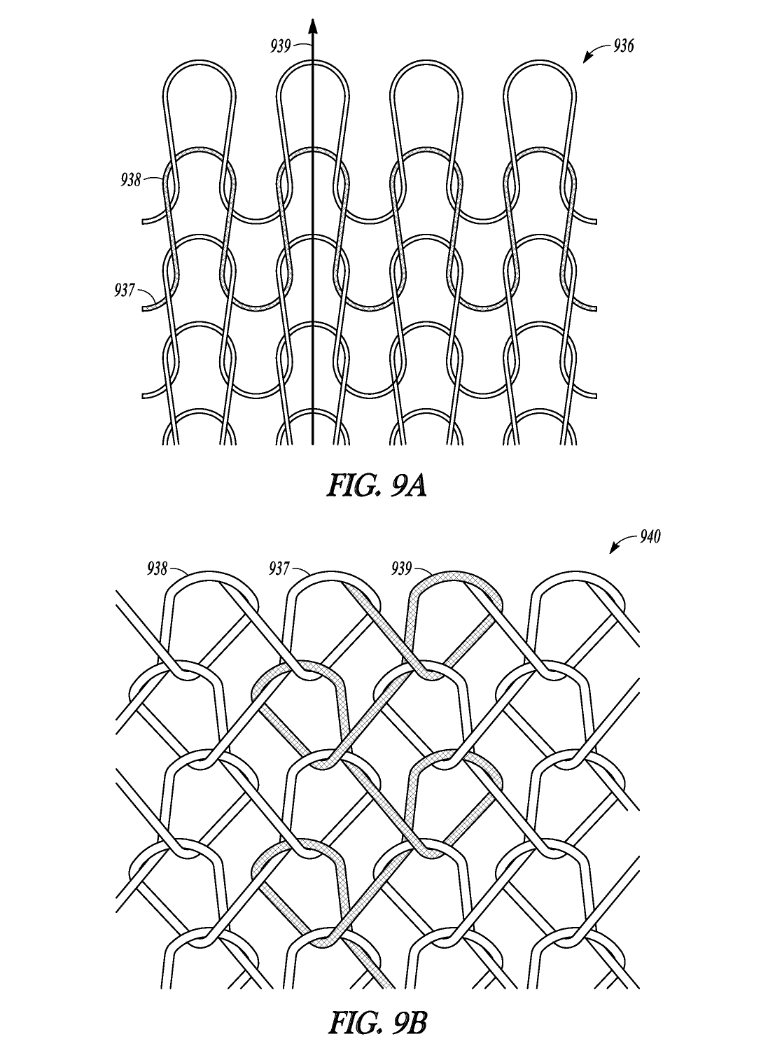

FIGS. 9A-B illustrate examples of knitted textile forms suitable for flexible containment devices in accordance with various examples of the present disclosure. Similar to weaving, knitting is a technique for producing a fabric made from fibers. In weaving, the fibers are straight, running parallel either lengthwise (warp fibers) or crosswise (weft fibers). By contrast, as illustrated in FIG. 9A, the fiber in knitted fabrics follows a meandering path or course 937, forming symmetric loops 938 (also called bights) symmetrically above and below the mean path of the fiber. There are two major varieties of knitting: weft knitting 936 and warp knitting 940 (see FIG. 9B). In weft knitting 936, the wales 939 are perpendicular to the course 937 of the fibers. In warp knitting, the wales 939 and courses 937 run roughly parallel. In weft knitting 936, the entire fabric may be produced from a single fiber, by adding stitches to each wale 939 in turn, moving across the fabric as in a raster scan. In warp knitting 940, as illustrated in FIG. 9B, one yarn can be required for every wale 939. In warp knitting 940, the wales 939 and courses 937 run roughly parallel The meandering loops 938 can be stretched easily in different directions, which can produce more elasticity than woven fabrics. As in the examples of the flexible containment devices above, a knitted flexible containment device can have fibers of various sizes and types in the same textile.

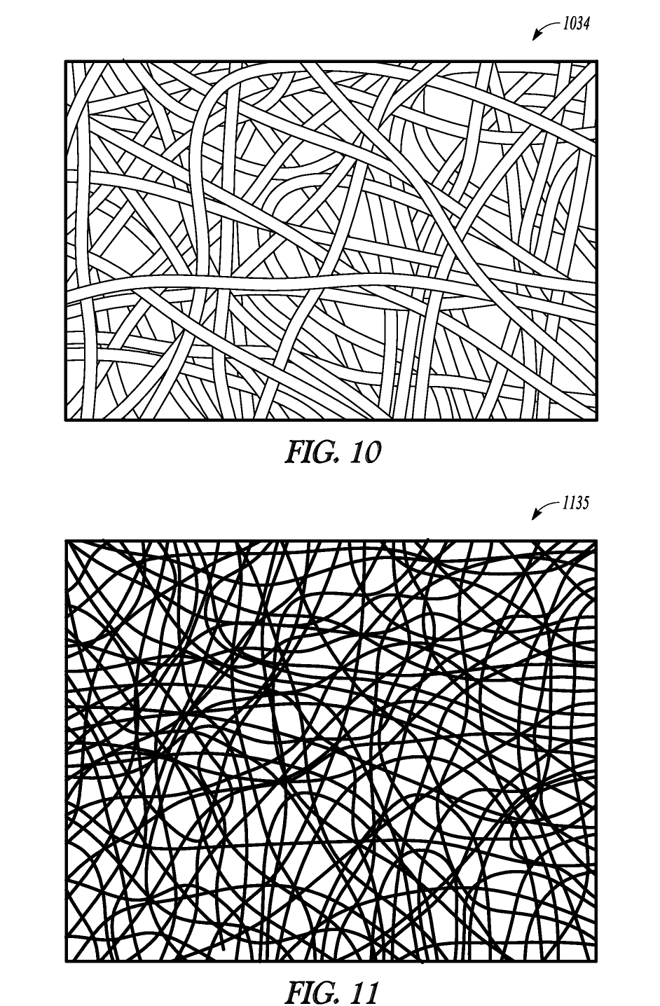

FIG. 10 illustrates a felt 1034 suitable for flexible containment devices in accordance with at least one example of the present disclosure. Felt 1034 is a non-woven textile that is produced by matting, condensing and pressing fibers. As in the examples of flexible containment devices described above, a felt flexible containment device can have fibers of various sizes and types in the same textile.

FIG. 11 illustrates an example of an electrospun material 1135 in accordance with at least one example of the present disclosure. Electrospinning uses an electrical charge to draw very fine (typically on the micro or nano scale) fiber from a liquid. Electrospinning shares characteristics of both electrospraying and conventional solution dry spinning of fibers. As in the examples of flexible containment devices described above, an electrospun flexible containment device can have fibers of various sizes and types in the same textile.

The fibers of the flexible containment devices described above can be coated with other materials either before the textile is formed or after the textile formation takes place. The coatings can perform functions such as inducing bone growth or retarding soft tissue growth. In various examples, the coatings can add strength, increase durability and bioabsorbability, provide a desired porosity, or allow for setting of a desired flexible containment device shape. In one exemplary application, one or more coatings inducing bone growth can be located on a bone facing surface of the flexible containment device and one or more coatings inducing soft tissue growth can be located on the opposite side, such as a side facing the gingiva. The textile types described above can be combined in the production of a flexible containment device. In an example, a knitted or braided form can have fibers woven through the braided or knitted fibers to provide added strength or durability. In another example, fibers of a textile form such as a woven, knitted or braided textile can be coated with electrospun fibers to provide properties promoting bone ingrowth or creating a textile that is impermeable to prevent soft tissue ingress into a desired bone growth area. To create a flexible containment device, any of the textile forms can be layered to form plies of material. Each ply can have particular functions or forms such as porosity, flexibility, strength, or stiffness. The whole textile or each ply individually can be treated with films or coatings to produce a desired surface chemistry, texture, or drug elution to promote, speed up, slow down, or inhibit soft tissue and/or bone tissue growth. Fiber and textile coatings can be formulated to provide wear resistance, non-stick, hydrophilic/hydrophobic, low friction, dielectric/conductive or corrosion resistant surface properties.

The flexible containment device can be formed with all or portions of the fabric being resorbable. The flexible containment device can be formed with all or portions of the fabric being biocompatible and configured to remain in place after a surgery without the need for later removal. The flexible containment device can be formed with all or portions of the textile having differing levels of permeability to perform functions such as allowing certain biological materials to pass through and block other biological materials.

### Ridge Splitting:

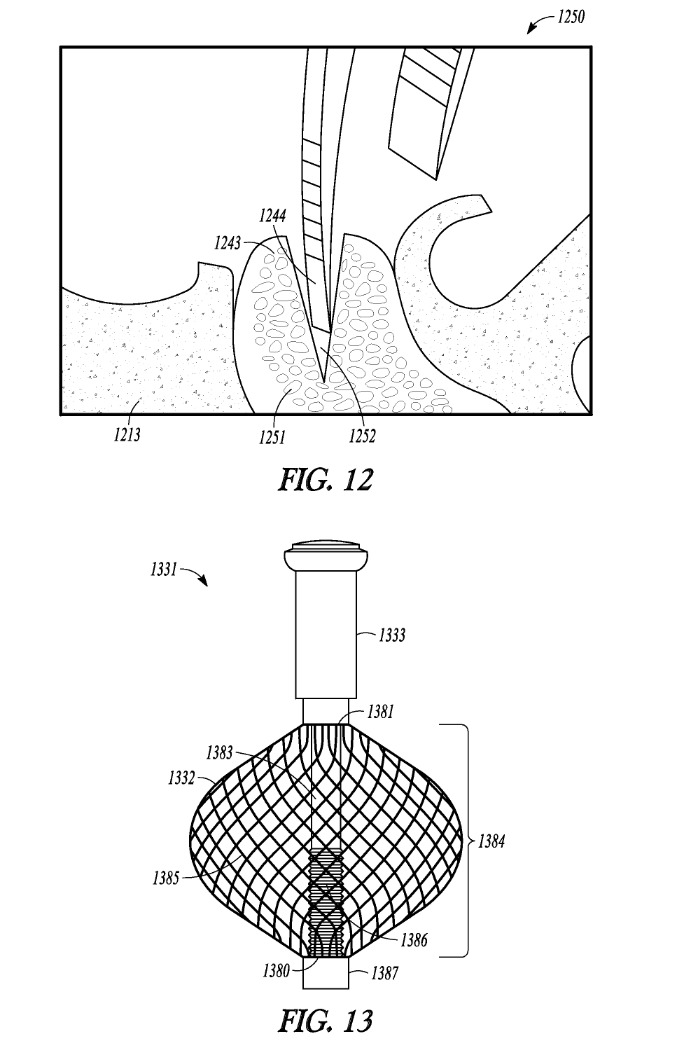

FIG. 12 illustrates an example of ridge splitting 1250 in accordance with at least one example of the present disclosure. In some cases, the thickness of a ridge 1243 of the mandible 1251 or maxillary bone is not large, strong or healthy enough to securely hold a dental implant. Additional bone can be created by cutting the gingiva 1213, splitting the ridge 1243, distracting the two halves with a distraction device 1244, and packing bone graft into a distraction area 1252 between the two halves.

A flexible containment device having any of the properties described above can be used to aid in placement of a bone graft in a ridge splitting procedure and in retaining vertical bone height. In addition to containing the graft and maintaining space for vertical bone, the flexible containment device can also provide the distraction required to separate the plates of the ridge or maintain a separation that was performed by a ridge-splitting device. The placement of the bone graft material into a non-permeable flexible containment device can provide the distraction. The device can be placed through one access incision rather than a full length incision typically used for current ridge splitting techniques, resulting in lower risk of infection and decreased pain and healing time. Due to the flexibility of this type of device, the surgical approach can also be oriented from lingual or facial surfaces of the ridge. The flexible containment device can be made non-permeable by multiple means such as the following:
1) The textile can be formed so as to be non-permeable to the bone graft while remaining permeable to blood. This example can be accomplished by providing a portion of the device with resorbable filaments to allow for greater porosity in the containment device if such porosity is needed at a later point in time.
2) Coating a permeable textile with a secondary non-permeable textile or film. The secondary material can be resorbable or non-resorbable.
3) Inserting a non-permeable balloon inside the flexible containment device to provide the distraction, and then removing the balloon prior to introducing the graft material. This non-permeable balloon can be a thin walled balloon similar to those used for angioplasty procedures.

### Sinus Lift:

In some cases of tooth loss, a bone ridge has resorbed toward the sinus cavity and/or the sinus cavity has resorbed toward the ridge, leaving too little bone to allow for implant placement. In these cases, a sinus lift can be performed to create additional bone to allow for implant placement. Complications for this procedure can include:
1) Infection at the incision in the gingival tissue or due to perforation of the Schneiderian membrane (sinus membrane). The motivation for creating a relatively large incision can be to visualize the membrane as it is being moved, verify no tears in the membrane have been created, treat tears that have been created in the membrane, and assure the bone graft is properly positioned.
2) Shifting of the bone graft, due to sudden movements, such as sneezing, before incorporation of the bone graft.

A flexible containment device having any of the characteristics described above can be used in a sinus lift procedure. The flexible containment device can be either continuous with a screw implant or can be attached to the screw implant. Such a device can allow an implant screw to be placed during the same procedure in which a bone graft is placed. The flexible containment device can be created using any one or combinations of the means described above.

A flexible containment device in accordance with the present disclosure can address the complications listed above. For example:
i. Infection—The outer surface of the flexible containment device can be configured such that it is smooth enough to protect a Schneiderian membrane during a graft insertion, limiting risk of membrane tears. The dental implant can be inserted into a surgical site and can remain permanently in place. The flexible containment device can contain the bone graft material during and after a graft placement. A smooth outer surface of the flexible containment device could be a resorbable polymer film designed to resorb at a time when the graft has integrated with the bone. Due to the reduced risk of damage to the sinus membrane and migration of the graft, a smaller incision can be used, reducing the risk of incision site infection and reducing healing time.
ii. Shifting of bone graft—As indicated above, the flexible containment device can be made impermeable to the graft material by a resorbable polymer membrane. The resorbtion rate can be tailored by altering the resorbable polymer. The polymer membrane can be created on the outer surface of a flexible containment device by such means as dip coating or electrospinning.

An example of a flexible containment device for sinus lift can include a metal braid with a resorbable polymer film outer coating. A technique for implanting a sinus lift device can include creating a small hole in the bone to access the sinus cavity. This access hole can be coincident with the hole for placement of the screw implant. The flexible containment device can then be introduced into the sinus cavity. As the bone graft is injected or packed in the flexible containment device, the sinus membrane will be lifted—no separate step is required for this as is required for current techniques. Bone graft or cement in the form of paste, granules or liquid can be packed or injected into the containment device. Materials can include, for example, autograft, allograft, or cements whose chemical reactions are initiated via mixing of chemicals or exposure to an energy source such as light. In addition to the flexible containment device, an endplate can be used to raise the membrane prior to introducing the graft/cement. The implant can be attached to the flexible containment device or placed at a later date.

FIG. 13 illustrates a braided flexible containment device 1331 in accordance with at least one example of the present disclosure that includes a braided portion 1332, having a distal end 1380 and a proximal end 1381. The containment device 1331 can include an outer longitudinal member 1333 and an inner longitudinal member 1383. The inner longitudinal member 1383 can be moved relative to the outer longitudinal member 1333 to alter a distance 1384 between the distal end 1380 and the proximal end 1381. As the distance 1384 is altered, the shape of the braided portion 1332 can change. As the distance 1384 is shortened, the braided portion 1332 will expand. If the distance 1384 is lengthened, the braided portion 1332 will narrow. In an example, the inner longitudinal member 1383 can include threads 1386. A nut portion 1387 can include mating female threading (not pictured). By rotating the threads 1386 of the the inner longitudinal member 1383 relative to the nut portion 1387 the distance 1384 can be altered. Such alteration in the shape of the braided portion 1332 can tailor such a containment device 1331 to a particular implant site as well as allow the containment device 1331 to be installed into a smaller incision and later expanded. Bone graft material can be pumped or placed into the spaces internal or external to the braided portion 1332. Bone graft material can be pumped through containment device 1331 including through passageways connecting a coronal end 1382 containment device 1331 with the internal space 1385 surrounded by the braided portion 1332. Such features can be used to attach an implant into an area of bone loss and provide stability and structure as a bone graft heals.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A flexible containment device comprising:
a textile material including at least one of a woven material, a braided material, a knit material, a felt material, and an electrospun material, wherein the textile material includes a plurality of biocompatible strengthening fibers configured to engage a bone graft material and configured to remain in a patient.

2. The flexible containment device of claim 1, wherein the textile material includes a woven material.

3. The flexible containment device of claim 1, wherein the textile material includes a braided material.

4. The flexible containment device of claim 1, wherein the textile material includes a knitted material.

5. The flexible containment device of claim 1, wherein the strengthening fibers are metal fibers and wherein the textile material includes the metal fibers in a range of about 20% to about 60% by weight.

6. The flexible containment device of claim 5, wherein the metal fibers comprise at least one of a stainless steel, titanium, titanium alloy, and nitinol.

7. The flexible containment device of claim 1, wherein the strengthening fibers are polymer fibers and wherein the textile material includes the polymer fibers in a range of about 20% to about 60% by weight.

8. The flexible containment device of claim 7, wherein the polymer fibers are comprised of at least one of polyether ether ketone (PEEK), polyethelene, poly(methyl methacrylate) (PMMA), polyester, and polytetrafluoroethylene (PTFE).

9. The flexible containment device of claim 1, wherein the strengthening fibers include metal fibers in a range of about 10% to about 40% by weight and polymer fibers in a range of about 10% to about 40% by weight.

10. The flexible containment device of claim 1, wherein the strengthening fibers include metal fibers in a range of about 20% to about 60% by weight and polymer fibers in a range of about 20% to about 60% by weight.

11. The flexible containment device of claim 1, wherein the fibers of the textile material comprise single strand fibers.

12. The flexible containment device of claim 1, wherein the strengthening fibers of the textile material comprise multi-strand fibers.

13. The flexible containment device of claim 1, wherein the textile material is configured to be placed in and remain in a mouth of the patient.

14. A flexible containment device comprising:
a textile material including at least one of a woven material, a braided material, a knit material, a felt material, and an electrospun material, wherein the textile material includes a plurality of strengthening fibers, a plurality of resorbable fibers and a plurality of biocompatible fibers configured to engage a bone graft material and configured to remain in a patient.

15. The flexible containment device of claim 14, wherein the textile material is configured to be placed in and remain in a mouth of the patient.

16. The flexible containment device of claim 14, wherein the plurality of strengthening fibers include at least one of stainless steel, titanium, titanium alloys, nitinol, polyether ether ketone (PEEK), polyethelene, poly(methyl methacrylate) (PMMA), polyester, and polytetrafluoroethylene (PTFE).

17. The flexible containment device of claim 14, wherein the plurality of resorbable fibers are comprised of at least one of poly-L-lactide (PLLA), polyglycolic acid (PGA), and hydrogels.

18. A method of bone grafting comprising:
cutting a gingival layer;
exposing a bone surface;
decorticating the bone surface;
packing bone graft material into the bone surface;
covering the bone graft material with a flexible containment device, the flexible containment device formed by at least one of braiding, weaving, knitting, felting and electrospinning, the flexible containment device further including a bone graft facing surface coated with a bone growth inducing substance; and
suturing the cut gingival layer.

19. The method of claim 18, wherein the flexible containment device is comprised of a plurality of metal fibers.

20. The method of claim 18, wherein the flexible containment device is comprised of a plurality of metal fibers and a plurality of polymer fibers.
